(19) United States
(12) Patent Application Publication
(10) Pub. No.: US 2005/0228359 A1
(43) Pub. Date: Oct. 13, 2005

(54) Method and apparatus for treating acute stroke

(76) Inventor: Aiden J. Doyle, Princeton, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/098,088

(22) Filed: Apr. 4, 2005

Related U.S. Application Data
(60) Provisional application No. 60/560,202, filed on Apr. 7, 2004.

(51) Int. Cl. A61M 31/00

(52) U.S. Cl. 604/509

(57) ABSTRACT

The invention provides a method and system for introducing cool fluid to a targeted treatment site, such as, for example, a stroke-affected brain hemisphere. In one approach, the method generally includes introducing a catheter into a branch artery of the external carotid artery, and introducing fluid into the ipsilateral internal carotid artery. In one approach, a balloon is used to occlude the external carotid artery during introduction of the fluid into the internal carotid artery.

### RELATED APPLICATIONS

This application claims the benefit of U.S. Provisional Application No. 60/560,202, filed Apr. 7, 2004, the contents of which are incorporated in their entirety herein by reference.

### BACKGROUND OF THE INVENTION

1. Field of the Invention

The present invention relates generally to the treatment of ischemic organs, such as the brain in the setting of transient ischemic attack (TIA) or stroke, and more specifically to methods and apparatus for delivering a cool fluid to the vascular territory of brain regions that are experiencing ischemia and/or acute stroke.

2. Description of the Related Art

Stroke is the third leading cause of death in the United States and is the leading disabling neurological disorder. A stroke or cerebrovascular accident (CVA) refers to a situation in which the blood supply to a brain region is interrupted by occlusion (an ischemic stroke) or by hemorrhage (a hemorrhagic stroke). A hemorrhagic stroke occurs when a blood vessel in the brain, an arteriovenous malformation, or a cerebral aneurysm ruptures, spilling blood into the spaces surrounding the brain cells.

Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Outcome following stroke is influenced by a number of factors, the most important being the nature and severity of the resulting neurologic deficit. Overall, less than 80% of patients with stroke survive for at least 1 month, and approximately 35% have been cited for the 10-year survival rates. Of patients who survive the acute period, up to 75% regain independent function, while approximately 15% require institutional care.

Hemorrhagic stroke accounts for 20% of the annual stroke population. Hemorrhagic stroke often occurs due to rupture of an aneurysm or arteriovenous malformation bleeding into the brain tissue, resulting in cerebral infarction. The remaining 80% of the stroke population are hemispheric ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. The internal carotid artery, commonly affected by atherosclerosis causing symptomatic occlusion in the arterial lumen, is often responsible for hemispheric ischemic stroke and generating thromboembolic material downstream to the distal cerebral vessels. Treatment of the occluded carotid artery in patients with stroke and TIA or for stroke prevention in patients with asymptomatic flow limiting carotid stenosis undergoing major cardiothoracic surgeries includes performing angioplasty, stent placement, or atherectomy on the occluded carotid artery. Unfortunately, placing instrumentation within a diseased carotid artery is associated with increased risk of ischemic stroke, since manipulation of an atheromatous plaque in the arterial wall often causes emboli to dislodge distally in the narrow cerebral arteries.

There are generally three treatment stages for stroke: (1) prevention; (2) therapy immediately after stroke; and (3) post-stroke rehabilitation. Therapies to prevent stroke are based on treating underlying risk factors. Acute stroke therapies try to stop a stroke while it is happening. Post-stroke rehabilitation is to overcome disabilities that result from stroke damage. While advances have been made in the area of acute stroke therapies (i.e., treatment stage 2), there exists a need for devices and methods for reducing the effect of strokes when they occur.

One acute stroke therapy involves inducing hypothermia of the body to protect the brain from injury. Hypothermia is believed to lower the metabolic demands in the penumbral area surrounding the necrotic or apoptotic core of the infarct. Cooling of the brain can be accomplished through whole body cooling to create a condition of total body hypothermia in the range of about 20° C. to about 30° C. Another acute stroke therapy involves inducing hypothermia of the head to protect the brain. For example, some physicians have immersed the patient's head in ice, or have used cooling helmets or head gear to achieve the same. It has been shown, however, that complications (e.g., arrhythmias and decreased cardiac output) can arise from total body cooling or cooling of the face and head only.

Selective organ hypothermia is a promising acute stroke therapy, and involves perfusing the targeted organ with a cold solution, such as saline or perflourocarbons. Challenges with this mode of therapy include avoiding excessive volume accumulation, temperature dilution by blood, and damage to tissue and vessels, particularly near the already traumatized targeted organ. There exists a need for a treatment that can be implemented soon after the onset of a stroke episode to salvage the brain cells surrounding the damaged brain cells.

### SUMMARY OF THE INVENTION

In accordance with one aspect of the embodiments described herein, there is provided a method of introducing cool fluid to a targeted treatment site. In one application, the treatment site comprises a stroke-affected brain hemisphere. In one approach, the method generally comprises: introducing a catheter into an opening in a branch artery of the external carotid artery that is ipsilateral to the hemisphere; advancing the catheter from the opening in the branch artery along the branch artery in a direction retrograde to physiologic blood flow; positioning an opening in said catheter at the bifurcation of the ipsilateral internal and external carotid arteries; and while temporarily occluding the external carotid artery, introducing fluid through the opening in said catheter such that the fluid flows into the ipsilateral internal carotid artery in a direction antegrade to physiologic blood flow.

In accordance with one aspect of the embodiments described herein, there is provided a method of introducing cool fluid to a targeted treatment site. In one application, the treatment site comprises a stroke-affected brain hemisphere. In one approach, the method generally comprises: introducing a catheter into an opening in a branch artery of the external carotid artery that is ipsilateral to the hemisphere; advancing said catheter from the opening in the branch artery along the branch artery in a direction retrograde to physiologic blood flow; positioning an opening in said catheter within the external carotid artery; and introducing fluid through the opening in said catheter such that the fluid flows in the external carotid artery in a direction retrograde to physiologic blood flow and then flows into the ipsilateral internal carotid artery in a direction antegrade to physiologic blood flow.

In accordance with one aspect of the embodiments described herein, there is provided a system for delivering cool fluid to a targeted treatment site. In some embodiments, the treatment site comprises a stroke-affected and/or ischemic brain region.

In some embodiments, the system generally comprises a catheter configured for use in the vessels of the head and neck regions, where the catheter extending between from a proximal end to a distal end, where the catheter comprises an inner lumen and a distally-located opening that is in communication with the lumen. The system further comprises a supply of cool fluid connected to the lumen near the catheter distal end for delivering cool fluid to a targeted vascular region. In some embodiments, the cool fluid comprises cold crystalloid solution.

In some embodiments, the system generally comprises a flexible, elongate catheter, the catheter having a proximal end and a distal end, the catheter comprising a lumen and a distal opening that is in communication with the lumen; a counterpulsation balloon mounted at or near the catheter's distal end; a controller in communication with the counterpulsation balloon, the controller configured to produce delivery of a quantity of a fluid through the catheter when the counterpulsation balloon is primarily in an inflation mode, the controller further configured to decrease or cease the delivery of the fluid through the catheter when the counterpulsation balloon is primarily in a deflation mode.

In some embodiments, the catheter is configured to be positioned in an external carotid artery of a human. Some embodiments further comprise a receptacle configured to supply the fluid to the catheter. In some embodiments, the supply comprises a refrigeration system configured to keep the fluid below 30° C.

In some embodiments, the system further comprises a counterpulsation balloon mounted near the catheter distal end, and a counterpulsation controller that is communication with the balloon.

### BRIEF DESCRIPTION OF THE DRAWINGS

FIG. 1 illustrates various great vessels of the systemic circulatory system.

FIG. 2 depicts normal cerebral circulation in the Circle of Willis.

FIG. 3 is a schematic representation of human head displaying the main arteries that branch from the external carotid artery and that supply blood to the head, face, and neck area.

FIG. 4 is a schematic representation of a common carotid artery in the head and neck of a patient.

FIG. 5 is a schematic representation of the branching of the external and internal carotid arties.

FIG. 6A illustrates a method of introducing cool fluid by positioning a small vessel catheter at the bifurcation of the common carotid artery into the external and internal carotid arteries.

FIG. 6B illustrates a method of introducing cool fluid by positioning a small vessel catheter in the external carotid artery.

FIG. 7A illustrates a method of introducing cool fluid by positioning a balloon catheter assembly at the bifurcation of the common carotid artery into the external and internal carotid arteries.

FIG. 7B illustrates a method of introducing cool fluid by positioning a balloon catheter in the external carotid artery.

FIG. 8 depicts one embodiment of a system for delivering cool fluid to a stroke-affected brain hemisphere.

FIG. 9 is a cross-sectional view of the catheter of the system in FIG. 8.

### DETAILED DESCRIPTION OF THE PREFERRED EMBODIMENT

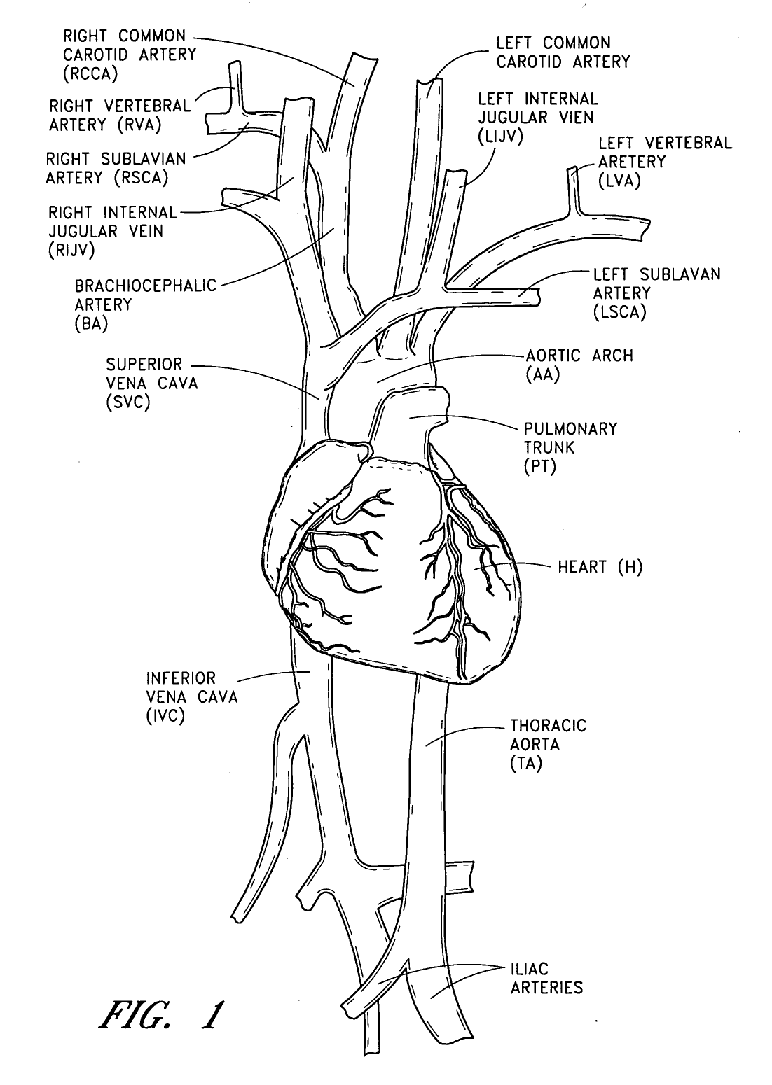

With reference to FIG. 1, oxygenated blood from the heart normally flows upwardly through the aortic arch and then downward to the lower portions of the body through the thoracic aorta. Three major arteries extend upwardly from the top of the aortic arch. The brachiocephalic, or inominate, artery branches into the right carotid artery and the right subclavian artery. In contrast, the left carotid artery and left subclavian artery extend directly from the aortic arch and do not have a common portion. Along with the vertebral arteries, the right and left common carotid arteries provide oxygenated blood to most parts of the head and neck. They ascend through the anterior neck just lateral to the trachea and are covered by relatively thin muscles. In addition to the carotid arteries, oxygenated blood is provided to the brain through the vertebral arteries, although to a significantly lesser extent.

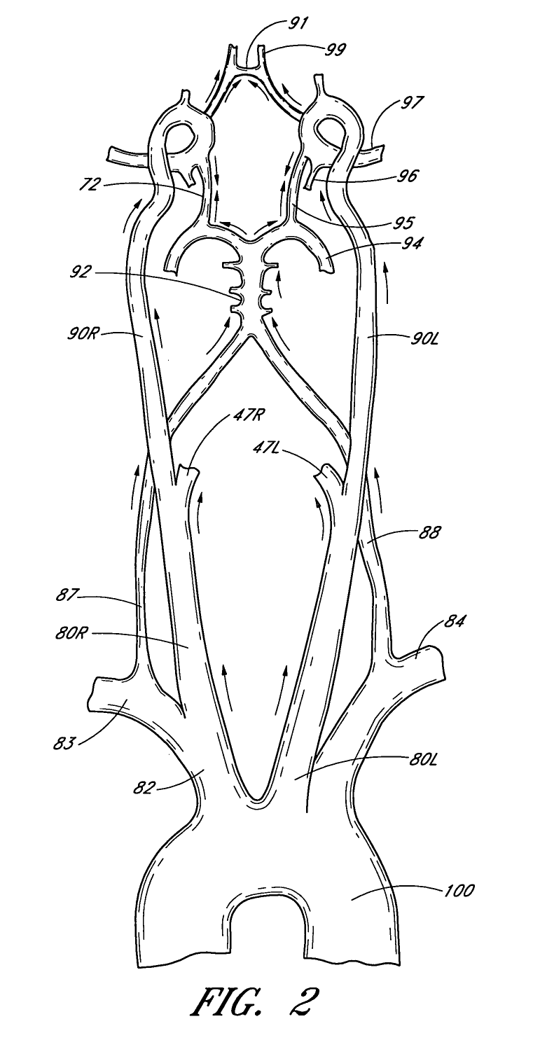

As FIG. 2 further illustrates aorta 100 gives rise to right brachiocephalic trunk 82, left common carotid artery (CCA) 80_{L}, and left subclavian artery 84. The brachiocephalic artery further branches into right common carotid artery 80_{R} and right subclavian artery 83. The left CCA gives rise to left internal carotid artery (ICA) 90_{L} which becomes left middle cerebral artery (MCA) 97 and left anterior cerebral artery (ACA) 99. Anteriorly, the Circle of Willis is formed by the internal carotid arteries, the anterior cerebral arteries, and anterior communicating artery 91 which connects the two ACAs. The right and left ICA also send right posterior communicating artery 72 and left posterior communicating artery 95 to connect respectively with right posterior cerebral artery (PCA) 74 and left PCA 94. The two posterior communicating arteries and PCAs, and the origin of the posterior cerebral from basilar artery 92 complete the circle posteriorly. The left CCA also gives rise to external carotid artery (ECA) 47_{L}, which branches extensively to supply most of the structures of the head except the brain and the contents of the orbit. The ECA also helps supply structures in the neck.

The cerebral circulation is regulated in such a way that a constant total cerebral blood flow (CBF) is generally maintained under varying conditions. For example, a reduction in flow to one part of the brain, such as in stroke, may be compensated by an increase in flow to another part, so that CBF to any one region of the brain remains unchanged. Also, when one part of the brain becomes ischemic due to a vascular occlusion, the brain compensates by increasing blood flow to the ischemic area through its collateral circulation via the Circle of Willis.

The internal carotid artery supplies the anterior part of the brain, the eye and its appendages, and sends branches to the forehead and nose. The external carotid artery begins opposite the upper border of the thyroid cartilage, and, taking a slightly curved course, passes upward and forward, and then inclines backward to the space behind the neck of the mandible, where it divides into the superficial temporal and internal maxillary arteries.

As illustrated in FIG. 2, the internal carotid arteries 80_{R}, 80_{L} has no branches in the neck. It ascends and enters the cranium through the carotid canal. Inside the cranium, it gives off the ophthalmic artery and trifurcates into the anterior cerebral artery, middle cerebral artery, and posterior communicating artery. The latter three arteries contribute to an important anastomosis in the brain, the Circle of Willis.

The external carotid artery usually has eight branches in the neck: superior thyroid artery, lingual artery, facial artery, ascending pharyngeal artery, occipital artery, posterior auricular artery, maxillary artery, and superficial temporal artery. The latter two could be considered a terminal bifurcation of the artery; the maxillary artery is the larger of the two.

FIG. 3 graphically depicts the network arteries branching from the external carotid artery 47 within the head. Superimposed upon this view are the spatial divisions of the human head. The posterior division F includes the back of the neck and extends upwardly behind the ear, as encompassed by the dashed lines 40 and 41. The ophthalmic area G, lying above the dashed lines 41 and 42, includes the top and frontal area of the scalp and head. The superficial cervical plexus H, defined by the dashed line 40 at the rear and the dashed line 43 at the front, includes the front and sides of the neck, the underside of the chin and diverges upwardly to include the ear. The mandibular area I, lying above the dashed line 43 and below the dashed line 44, generally includes the lower jaw and the temple area and extends upwardly to the ophthalmic area G. The maxillary area J, lying between the mandibular area I and the ophthalmic area G as defined by the dashed lines 44 and 42, respectively, generally includes the upper jaw and a narrow upward extension lying between the temple and the eye.

The external carotid artery 47 supplies the superficial parts of the head, face and neck. The occipital artery 48 is the first branch of the external carotid artery 47 as it delivers blood in the direction of the arrow K from the heart to the head. The occipital artery 48 supplies the scalp and the back of the head within the lower portion of the posterior division F. The sternocleidomastoid artery 49 branches from the occipital artery to deliver blood within the superficial cervical plexus H below and behind the ear. The superficial temporal artery 50 is an extension of the external carotid artery 47 which extends upwardly immediately in front of the ear. Immediately above the ear, the superficial temporal artery 50 subdivides into two primary branches: a first branch, the posterior temporal branch 51, which supplies the rearward portion of the ophthalmic area G and the upper portion of the posterior division F, and the anterior temporal branch 52 which supplies blood to the upper frontal portion of the ophthalmic area G. The superficial temporal artery 50 and the lower portion of the posterior temporal 51 and the lower portion of the anterior temporal 52 also supply blood to the upper portion of the mandibular area I and the maxillary area J.

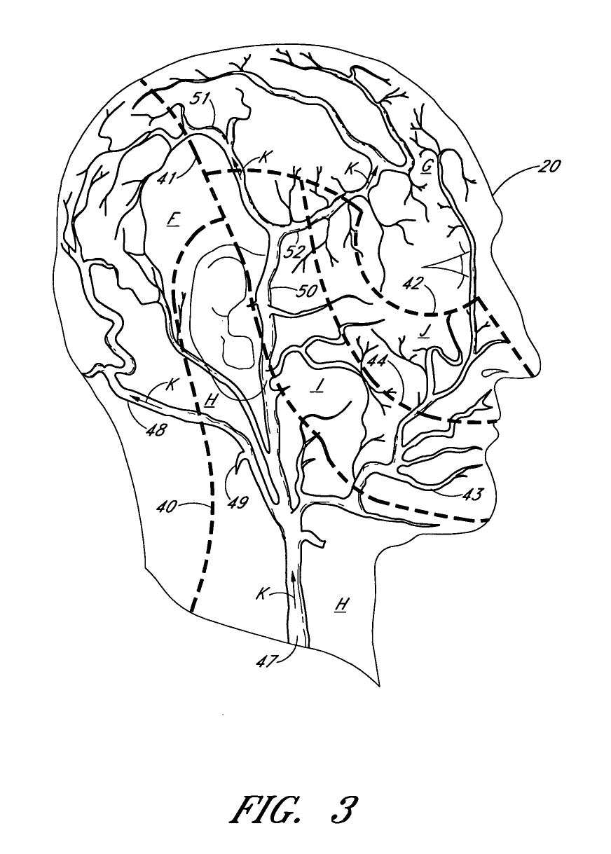

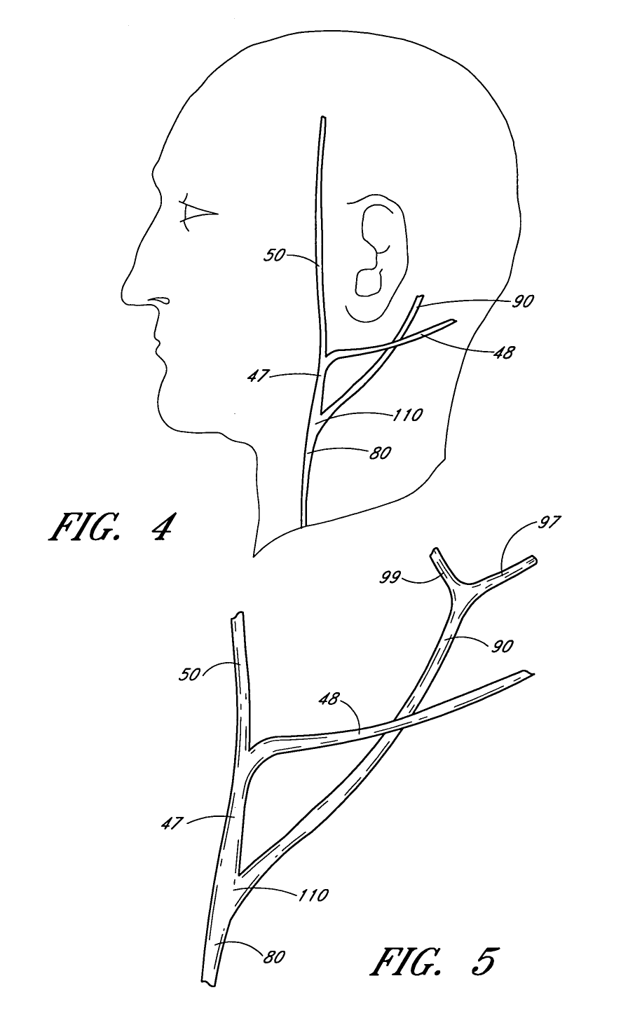

With reference to FIGS. 3 and 4, at approximately the level of the third cervical vertebra, the common carotid 80 branches into the internal carotid artery 90 and the external carotid artery 47. The external carotid artery 47 branches into numerous arteries in the head regions, including, for example, the external occipital artery 48 and the superficial temporal artery 50. FIG. 5 provides a schematic representation of the branching of the common carotid artery 80 into external carotid artery 47 and the internal carotid artery 90 at the junction or bifurcation 110.

As explained above, one promising acute stroke therapy involves perfusing the blood-deprived brain region with a cool solution to induce hypothermia in the brain region, thereby mitigating the effect of the stroke in the brain region. Disclosed herein is a method of selectively inducing hypothermia in the brain hemisphere where the stroke has occurred.

In accordance with one aspect of the embodiments described herein, there is provided a method for treating stroke by introducing cooled fluid into the a brain hemisphere that has been blood-deprived as a result of the stroke. In one approach, the method for introducing cooled fluid generally comprises: introducing a catheter into a branch artery of the external carotid artery that is ipsilateral to the brain hemisphere; advancing the catheter along the branch artery in a direction retrograde to physiological blood flow; and introducing cool fluid through the catheter opening such that the fluid flows into the ipsilateral internal carotid artery in a direction antegrade to physiologic blood flow.

Short of placing a catheter dripping cold crystalloid solution directly in the vascular territory of an infarct associated with a CVA, one preferred approach involves placing a catheter in the external carotid artery at the bifurcation of the internal carotid artery, and infusing a cool fluid (e.g., cooled saline) through the internal carotid ipsilateral to the CVA.

With reference to FIGS. 3 and 4, in one exemplary approach, the external carotid artery 47 is accessed through the superficial temporal artery 50. In another exemplary approach, the external carotid artery is accessed through the external occipital artery 48, particularly if there is a concern about a superficial temporal artery/middle cerebral artery anastomosis or potential bypass. Either of these vessels 48, 50 are readily cannulated and lead directly to the carotid bifurcation.

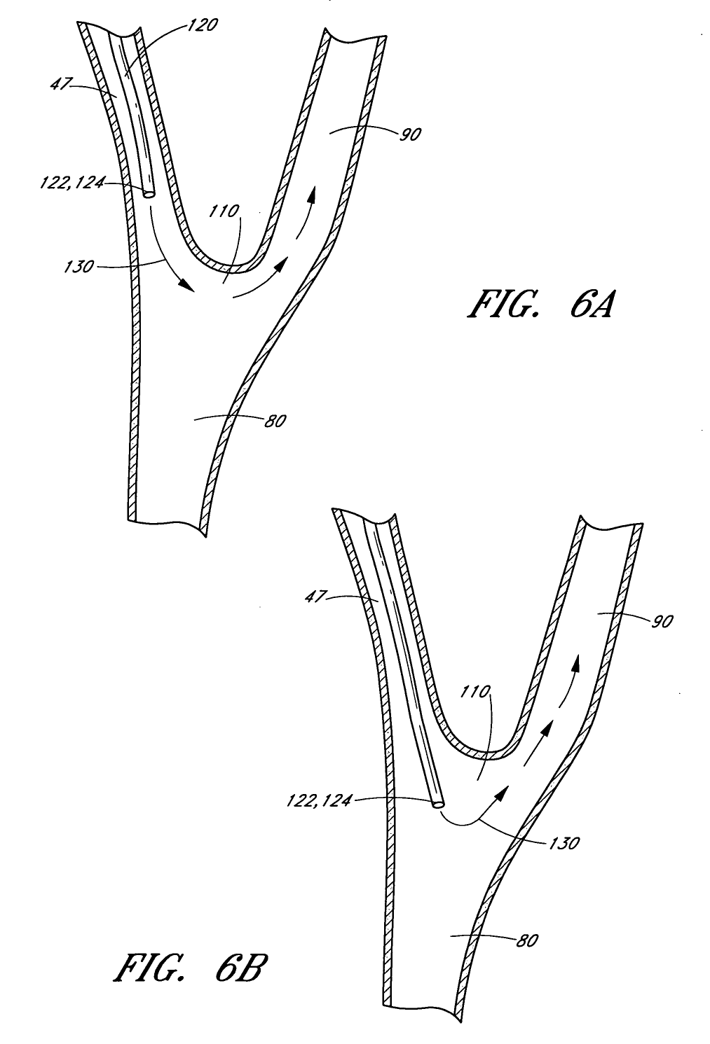

With reference to FIG. 6A, in one approach, a percutaneously insertable catheter 120 is introduced in any known suitable branch artery of the external carotid artery 47 that is ipsilateral to the stroke-affected brain hemisphere. Suitable branch arteries include, for example, the occipital artery 48, the sternocleidomastoid artery 49, the superficial temporal artery 50, the posterior temporal branch 51, the anterior temporal branch 52, etc. In another approach, the 120 is introduced in the lower branch of the external carotid artery 47, closer to the bifurcation 110 of the ipsilateral external and internal carotid arteries 47, 90.

After the external carotid artery 47 has been catheterized, the catheter 120 is the advanced toward the bifurcation 110 of the ipsilateral external and internal carotid arteries 47, 90, in a direction that is retrograde to physiological blood flow. The catheter 120 is positioned so that the catheter distal end 122, along with the catheter opening 124 near the distal end 122, is at or near the bifurcation 110. The catheter 120 is preferably advanced and positioned using any known suitable visualization technique, such as, for example, ultrasound imaging, fluoroscopy, radiography, etc.

With continued reference to FIG. 6A, in one approach, the catheter distal end 122 is positioned at the bifurcation 110. After the catheter 120 has been positioned near the bifurcation 110, a cool fluid 130 is introduced through the catheter opening 124 and into the ipsilateral internal carotid artery 90 in a direction antegrade to physiologic blood flow, thereby delivering cool fluid 130 to stroke-affected regions in the ipsilateral brain hemisphere.

Because only the ipsilateral hemisphere is being cooled, a reduced volume of cool fluid is needed to cool the targeted hemisphere, thereby resulting in reduced global or whole body cooling. Global cooling is also reduced due to the second pass of the infused cool fluid through the cardiopulmonary circulation.

With reference to FIG. 6B, in one approach, the catheter 120 is introduced into a branch of the external carotid artery 47 and advanced toward but just short of the bifurcation 110, leaving a short distance between the catheter distal end 122 and the bifurcation 110. After the distal end 122 has been positioned near the bifurcation 110, a cool fluid 130 is introduced through the catheter opening, 124 and into the external carotid artery 47 in a direction retrograde to physiologic blood flow. The cool fluid 130 is preferably pushed or pumped into external carotid artery 47 with enough pressure to overcome the blood pressure in the external carotid artery 47, so that the cool fluid 130 is redirected into ipsilateral internal carotid artery 90 in a direction antegrade to physiologic blood flow, thereby delivering cool fluid 130 to stroke-affected regions in the ipsilateral brain hemisphere.

In another approach, the catheter 120 is introduced into a branch of the external carotid artery 47 and advanced beyond the bifurcation 110. After the distal end 122 has been positioned beyond the bifurcation 110, a cool fluid 130 is introduced through the catheter opening 124, and into ipsilateral internal carotid artery 90 in a direction antegrade to physiologic blood flow, thereby delivering cool fluid 130 to stroke-affected regions in the ipsilateral brain hemisphere.

In one approach, the cool fluid 130 comprises normal saline that is cooler than the patient's body temperature. In some approaches, the fluid 130 has a temperature in the range of about 0° C. to about 30° C. In some approaches, the fluid 130 has a temperature of about 5° C. to about 20° C.

In one exemplary approach, the fluid 130 comprises isosmolar crystalloid or colloid solution, such as iced saline. In another approach, the fluid 130 comprises a mixture of cool saline and a stroke-treating medication, such as an antithrombotic, anticoagulant, thrombolytic, or antiplatelet drug.

In accordance with one aspect of the embodiments described herein, there is provided a method of delivering cool fluid to a stroke-affected or ischemic brain hemisphere, comprising: introducing a catheter assembly into a branch artery of the external carotid artery that is ipsilateral to the affected brain hemisphere; advancing the assembly along the branch artery in a direction retrograde to physiological blood flow; positioning an opening in the assembly at the bifurcation of the ipsilateral internal and external carotid arteries; and while temporarily occluding the external carotid artery, introducing cool fluid through the opening such that the fluid flows into the ipsilateral internal carotid artery in a direction antegrade to physiologic blood flow.

As explained below, in one approach, washout of the cool fluid can be prevented by a synchronized counterpulsation balloon that blocks the external carotid as the saline cycles at a rate that is matched or close to the patient's cardiac cycle (e.g., 30-60 times a second). With enough pressure from the infuser pump, it is possible for the infused cool fluid to overcome the blood pressure near the common carotid artery bifurcation and circulate through the internal carotid artery, and thus to the vascular territory of the infarction. Deflation of the balloon results in the reestablishment of blood flow up through the external carotid artery, thereby washing up any clot formation that may occur. Clot reduction can also be reduced through the use of a known suitable medication that prevents the formation of clots (e.g., carefully administered doses of heparin).

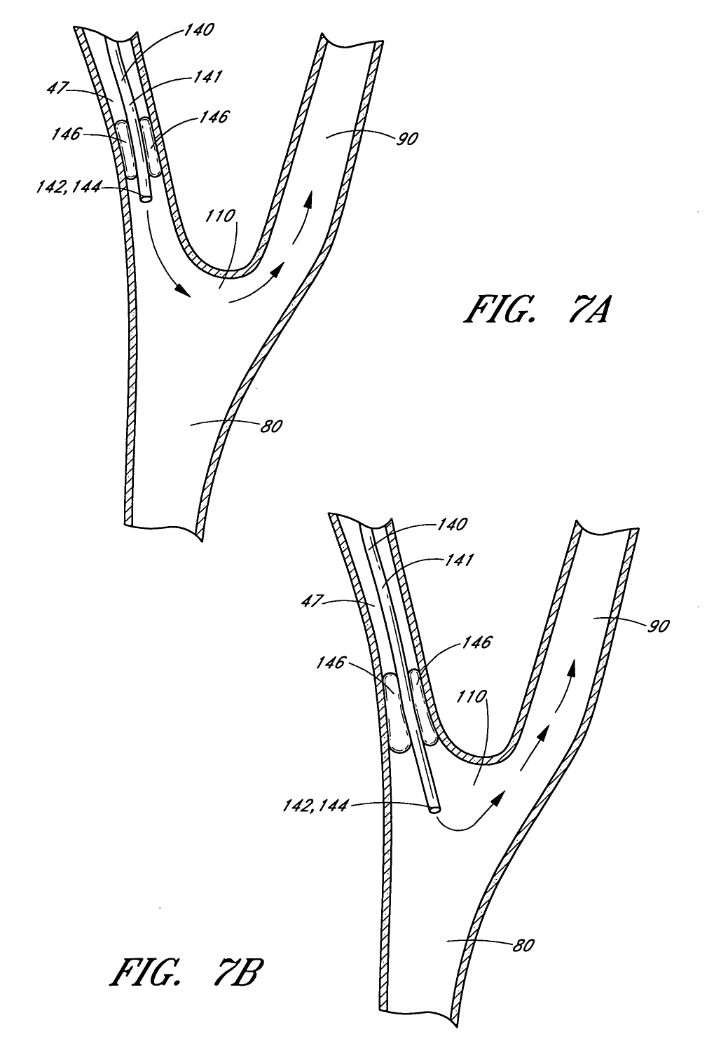

With reference to FIG. 7A, in one approach, a balloon catheter assembly 140 is percutaneously introduced into a branch of the external carotid artery 47 and advanced toward but just short of the bifurcation 110, leaving a short distance between the catheter distal end 142 and the bifurcation 110. After the distal end 142 has been positioned at or near the bifurcation 110, the external carotid artery 47 is temporarily occluded by distally expanding the balloon 146 of the catheter assembly 140, thereby temporarily blocking blood flow through the external carotid artery 47. In one approach, expansion of the balloon 146 partially, temporarily blocks blood flow through artery 47. In another approach, expansion of the balloon 146 completely, temporarily blocks blood flow through artery 47.

With continued reference to FIGS. 7A and 7B, while temporarily occluding blood flow through the external carotid artery 47, a cool fluid 130 is introduced through the catheter opening 144, and into the external carotid artery 47 in a direction toward the bifurcation 110 and into ipsilateral internal carotid artery 90 in a direction antegrade to physiologic blood flow, thereby delivering cool fluid 130 to stroke-affected regions in the ipsilateral brain hemisphere. Because expansion of the balloon 146 occludes blood flow in the external carotid artery 47, less pressure is required to push cool fluid 130 into the ipsilateral internal carotid artery 90.

The distal end 142 of the catheter assembly 140 can be placed at number of suitable positions near the bifurcation 110. In one approach, shown in FIG. 7A, the catheter distal end 142 is positioned at the bifurcation 110. In another approach, shown in FIG. 7B, the catheter distal end 142 is positioned within the external carotid artery 47. In yet another approach, the catheter distal end 142 is positioned beyond the bifurcation 110. As explained below, for each of the approaches inflation of the balloon 146 and introduction of cool fluid 130 at or near the bifurcation 110 occurs during diastole, so that the fluid 130 enters the internal carotid artery 90.

In one approach, the balloon 146 is phasically pulsed in counterpulsation to the patient's cardiac cycle. The catheter assembly 140 is connected a controller 160 that receives the patient's electrocardiogram (ECG). In response to the ECG signal, the controller 160 causes the balloon 146 to be inflated during diastole (when the heart muscle is relaxed) and deflated during isometric contraction or early systole. Cool fluid 130 is introduced through the catheter opening 144 during diastole, when the balloon 146 is inflated, which facilitates delivery of the cool fluid 130 into the ipsilateral internal carotid artery 90. The cool fluid 130 is introduced at the bifurcation of the external and internal carotid arteries 47, 90. Because the balloon 146 blocks entry into the external carotid artery 47, blood from the common carotid artery 80 and cool fluid 130 from the catheter opening 144 are shunted into the internal carotid artery 90.

In accordance with one aspect of the embodiments described herein, there is provided a system for delivering cool fluid to a stroke-affected brain hemisphere. In some embodiments, the system comprises a flexible, elongate catheter extending between a proximal end and a distal end, a counterpulsation balloon mounted near the catheter distal end, a counterpulsation controller that is in communication with the balloon, and a supply of cool fluid connected to the catheter proximal end.

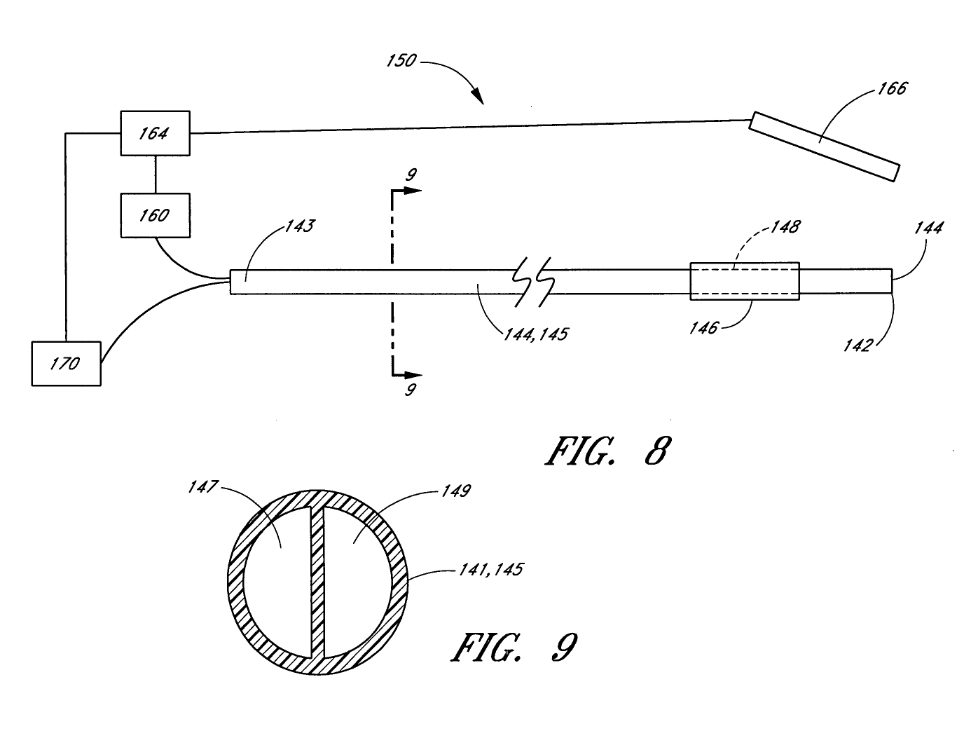

With reference to FIGS. 8 and 9, in some embodiments, the system 150 for delivering cool fluid 130 to a stroke-affected brain hemisphere comprises a catheter assembly 140, which comprises a small vessel catheter 141 and a counterpulsation balloon 146. The small vessel catheter 141 comprises an elongate, flexible, tubular body 145 that extends from a proximal end 143 to a distal end 142. The catheter 141 comprises a cool fluid lumen 149 that extends from the proximal end 143 to the distal end 142, and that is in communication with a catheter opening 144 near the distal end 142. The catheter 141 comprises an inflation lumen 147 and one or more port(s) 148 that allow communication between the lumen 147 and the inside of the balloon 146. The small vessel catheter 141 is dimensioned for use in the targeted vascular region. In one application, the targeted vascular region comprises the smaller vessels of the patient's body, such as, for example, the arteries which supply blood from the heart to the head, face, and neck areas.

The tubular body 145 and other components of the catheter 141 can be manufactured in accordance with any of a variety of techniques known in the catheter manufacturing field. Suitable material dimensions can be readily selected taking into account the natural and anatomical dimensions of the treatment site and of the desired percutaneous access site. The catheter 141 is preferably constructed from a biocompatible and flexible material (e.g., polyurethane, polyvinyl chloride, polyethylene, nylon, etc.). In one exemplary embodiment, the catheter 141 is 5 F and about 100 cm to about 200 cm in length to facilitate placement in the cerebral vasculature. Examples of suitable small vessel catheters can be found in U.S. Pat. No. 4,995,862, issued Sep. 11, 1990, titled CATHETER AND CATHETER/GUIDE WIRE DEVICE, the content of which is incorporated in its entirety herein by reference. The actual dimensions of a device constructed according to the principles of the embodiments described herein can vary outside of the any ranges listed herein without departing from the principles.

Preferably, the gas used to inflate the balloon 146 is carbon dioxide (which has fewer consequences in the rare event of a balloon bursting), helium (which has the fastest ability to travel or diffuse), or the like.

With continued reference to FIG. 8, the counterpulsation balloon 146 is any suitable expandable member and is similar to the balloon of an intra-aortic balloon pump (IABP) that has been adapted for use in vessels that supply blood to the head and neck regions. The counterpulsation balloon 146 is connected to an IABP-type, counterpulsation pump 160 which pumps an inflation fluid 162 (e.g., helium, carbon dioxide, etc.) into and out of the counterpulsation balloon 146 at specific times in relation to cardiac cycle of the patient.

The balloon 146 can be of the same material as currently in use in intra-aortic balloon pumps. The balloon 146 can be formed by dip molding on an appropriately shaped mandrel, with a hydrophilic coating. One specific commercially available polyurethane which has proven satisfactory in such balloons, including those used in practicing this invention, is sold by B.F. Goodrich under the designation Estane 58810. The balloon 146 is preferably made by dip casting on a mandrel having a shape corresponding to the inflated unstretched shape of the balloon. The dipping speed and time are adjusted to produce thin-walled balloons.

The bond between the balloon 146 and the catheter 141 is preferably airtight and effected in a manner to minimize the diametral dimension build-up of the catheter assembly 140. In some embodiments, balloon 146 and the catheter 141 are attached to each other via pressure-bonding with radiofrequency heating to a temperature approximating the melting temperature of the materials to enhance the bond and provide even greater control and to minimize the final outer dimensions in these bonding areas.

With continued reference to FIG. 8, a control apparatus 164 is connected to the counterpulsation pump/controller 160. The controller apparatus 164 drives and controls the counterpulsation pump 160, which in turn determine adjusts the inflation/deflation state of balloon 146. The control apparatus 164 typically comprises a logic computer system that receives data on the patient's ECG signal, arterial waveform, and/or an intrinsic pump rate, and that adjusts the counterpulsation pump rate on this data. In some embodiments, the inflation phase of the control apparatus 164 is triggered by the R wave of the patient's ECG signal. Balloon inflation is typically set to start in the middle of the T wave and to deflate prior to the ending QRS complex. It will be understood, however, that the control apparatus 164 can be selected and adjusted in any known suitable manner to achieve inflation of the balloon 146 during diastole and deflation during systole, or with an appropriate delay to cause fluid injection through a catheter to coincide with run-off of blood flow in a carotid or other artery

Because peripheral temperature measurements are often not accurate indicators of temperatures at particular locations, such as the CVA-affected brain hemisphere, it can be advantageous to implement temperature probes into the system 150. With continued reference to FIG. 8, in some embodiments, the system 150 comprises a temperature probe 166 placed in or near the affected hemisphere. The probe 166 is in communication with the control apparatus 164. The probe 166 can comprise any known suitable temperature monitoring device, such as, for example, a digital thermometer.

In some embodiments, the system 150 comprises a control apparatus 164 that performs intra-carotid-artery counterpulsation concurrently with the inducement/maintenance of hypothermia, the specifics of which are described in U.S. Pat. No. 6,800,068, issued Oct. 4, 2004, titled INTRA-AORTIC BALLOON COUNTERPULSATION WITH CONCURRENT HYPOTHERMIA, the content of which is incorporated in its entirety herein by reference.

A supply 170 of cool fluid 130 is attached to the distal end of the cool fluid lumen 149, and stores the cool fluid 130 that is pumped through the lumen 149, through the opening 144, and ultimately into the internal carotid artery 90. As explained above, in some embodiments, the cool fluid 130 comprises cold crystalloid solution. The supply 170 preferably has a cooling system (e.g., refrigerator, ice, etc.) that keeps the fluid 130 cool. The supply 170 preferably has a pumping mechanism or infuser pump for pushing the fluid 130 through the lumen 149 and the opening 144.

In some embodiments, shown in FIG. 8, the supply 170 is in communication with the control apparatus 164. The supply 170 receives a trigger or other control signal from the control apparatus 164 which activates the pumping mechanism, and thereby causes cool fluid 130 to be delivered through the catheter opening 144. In another embodiment (not illustrated), the supply 170 receives a control signal from a second control apparatus that is independent of control apparatus 164. The second control apparatus can comprise, for example, a button, switch, or peddle type of device that is controlled by the physician to initiate/stop the delivery of cool fluid 130 through the catheter lumen 149.

In accordance with one aspect of the embodiments described herein, there is provided a stroke treatment system comprising a flexible elongate catheter extending between a proximal end and a distal end, and a supply of cool fluid connected to the catheter proximal end. In some embodiments, the catheter is a small vessel catheter dimensioned for use in the vessels of the head and neck regions. In some embodiments, the stroke treatment system is similar to the system shown in FIG. 8, but lacks a balloon near the distal end of the catheter.

The methods and systems described herein relate to treating stroke. It will be noted, however, that these methods and systems can be adapted for the treatment of any number of medical conditions, such as, for example, focal contusions/hematomas associated with closed head injuries or the like. The methods and systems described herein can also be adapted for use in high-risk patients undergoing coronary artery bypass surgery. The methods and systems described herein can also be adapted for use in high-risk neurological procedures involving deep hypothermic arrest.

While the present invention has been illustrated and described with particularity in terms of preferred embodiments, it should be understood that no limitation of the scope of the invention is intended thereby. Features of any of the foregoing methods and devices may be substituted or added into the others, as will be apparent to those of skill in the art. The scope of the invention is defined only by the claims appended hereto. It should also be understood that variations of the particular embodiments described herein incorporating the principles of the present invention will occur to those of ordinary skill in the art and yet be within the scope of the appended claims.

1. A method of introducing fluid to a brain hemisphere in a mammal, the method comprising:
introducing a catheter assembly into an opening in a branch artery of the mammal's external carotid artery that is ipsilateral to the hemisphere;
advancing said catheter assembly from the opening in the branch artery along the branch artery in a direction retrograde to physiologic blood flow in the branch artery;
positioning an opening in said catheter assembly at or near the bifurcation of the mammal's internal and external carotid arteries that are ipsilateral to the hemisphere;
obstructing the external carotid artery at least partially; and
while at least partially obstructing the external carotid artery, introducing fluid through the opening in said catheter assembly such that the fluid flows into the mammal's ipsilateral internal carotid artery in a direction antegrade to physiologic blood flow in the ipsilateral internal carotid artery.

2. The method of claim 1, wherein said fluid is cooler than body temperature.

3. The method of claim 1, wherein said fluid has a temperature of about 0° C. to about 30° C.

4. The method of claim 3, wherein said fluid has a temperature of about 5° C. to about 20° C.

5. The method of claim 1, wherein the step of introducing fluid is performed during diastole of the mammal's cardiac cycle.

6. The method of claim 1, wherein the catheter assembly comprises a counterpulsation balloon.

7. The method of claim 6, wherein the step of temporarily occluding the external carotid artery comprises inflating the counterpulsation balloon.

8. The method of claim 7, wherein the step of inflating the counterpulsation balloon is performed during diastole.

9. The method of claim 1, wherein the fluid comprises a drug.

10. A method of introducing fluid to a brain hemisphere in a mammal, the method comprising:
introducing a catheter assembly into an opening in a branch artery of the mammal's external carotid artery that is ipsilateral to the hemisphere;
advancing said catheter assembly from the opening in the branch artery along the branch artery in a direction retrograde to physiologic blood flow;
positioning an opening in said catheter assembly within the external carotid artery; and
introducing fluid through the opening in said catheter assembly such that the fluid flows in the external carotid artery in a direction retrograde to physiologic blood flow and then flows into the mammal's ipsilateral internal carotid artery in a direction antegrade to physiologic blood flow.

11. A method for introducing fluid into a brain hemisphere in a mammal, the method comprising:
introducing a catheter assembly into an opening in a branch artery of the mammal's external carotid artery that is ipsilateral to the hemisphere;
advancing said catheter assembly from the opening in the branch artery along the branch artery in a direction retrograde to physiologic blood flow; and
introducing fluid through the opening in said catheter assembly such that the fluid flows into the mammal's ipsilateral internal carotid artery in a direction antegrade to physiologic blood flow.

12. A system for treating an ischemic organ, the system comprising:
a flexible, elongate catheter, the catheter having a proximal end and a distal end, the catheter comprising a lumen and a distal opening that is in communication with the lumen;
a counterpulsation balloon mounted at or near the catheter's distal end;
a controller in communication with the counterpulsation balloon, the controller configured to effect delivery of a quantity of a fluid through the catheter when the counterpulsation balloon is primarily in an inflation mode, the controller further configured to decrease or cease the delivery of the quantity of the fluid through the catheter when the counterpulsation balloon is primarily in a deflation mode.

13. The system of claim 12, wherein the catheter is configured to be positioned in an external carotid artery of a human.

14. The system of claim 12, further comprising a receptacle configured to supply the fluid to the catheter.

15. The system of claim 14, further comprising a refrigeration system in thermal communication with the receptacle, the refrigeration system configured to keep the fluid below 30° C.

16. The system of claim 12, wherein the controller is further configured to effect delivery repeatedly of quantities of fluid through the catheter during respective periods of inflation of the counterpulsation balloon, and the controller is further configured to cease or decrease delivery repeatedly of said quantities of fluid through the catheter during respective periods of deflation of the counterpulsation balloon.

17. A system for treating stroke in a patient, the system comprising:
first means for delivering fluid to an artery in the head of the patient;
second means for at least partially obstructing the artery, said second means being coupled to said first means;
third means for controlling delivery of a quantity of a fluid through the first means;
wherein said third means is in communication with said second means, such that said delivery of the fluid through the first means occurs when the second means is at least partially obstructing the artery, and said delivery of the fluid through the first means decreases or ceases when the second means is not at least partially obstructing the artery.
